(19) United States
(12) Patent Application Publication
(10) Pub. No.: US 2011/0066073 A1
(43) Pub. Date: Mar. 17, 2011

(54) BIOPSY DEVICE WITH ACOUSTIC ELEMENT

(76) Inventors: Stein Kuiper, Eindhoven (NL); Bernardus Hendrikus Wilhelmus Hendriks, Eindhoven (NL); Mareike Klee, Eindhoven (NL); Szabolcs Deladi, Eindhoven (NL); David Maresce, Rotterdam (NL)

(21) Appl. No.: 12/995,195

(22) PCT Filed: May 29, 2009
(86) PCT No.: PCT/IB2009/052270

§ 371 Date: Nov. 30, 2010

(30) Foreign Application Priority Data
Jun. 12, 2008 (EP) 08158086.2

(51) Int. Cl. A61B 10/00

(52) U.S. Cl. 600/562

(57) ABSTRACT

The invention relates to a biopsy device, particularly a biopsy device comprising a shaft with a transducer element for providing information about acoustic properties of a material to be analysed, a system of positioning a biopsy device and a method for positioning a biopsy device. The biopsy device may be adapted to take biopsies of different regions of the 5 human body for excluding or detecting abnormalities as cancerous lesions. The biopsy device may be used to measure acoustic properties of the material while inserting the tip portion of the biopsy device into the material to be analysed. The biopsy device may further allow measurement based on elastography.

### FIELD OF THE INVENTION

The invention generally relates to a biopsy device, particularly a biopsy device for providing information about acoustic properties of a material to be analysed, a system for positioning a biopsy device and a method for positioning a biopsy device.

### TECHNOLOGICAL BACKGROUND

For a correct diagnosis of cancer, usually biopsies are taken. This can either be done via the lumen of an endoscope or via biopsy needles. For example, needle biopsy is used to take biopsies from the prostate via the rectum. In order to find the correct position for taking the biopsies, various imaging modalities can be used such as X-ray, MRI and ultrasound. In case of prostate cancer, in most cases the needle is guided by an ultrasound probe that is inserted into the rectum.

Although helpful, these methods of guidance are far from optimal. There are two major problems directly related to the biopsy: The resolution is limited and, furthermore, these imaging modalities can in most cases not discriminate between benign and malignant tissue.

As a result, it is not known for certain whether the biopsy is taken from the correct position inside the tissue that should be analysed. Physicians take biopsies almost blindly and even if after inspection of the tissue no cancer cells are detected, they do not know for certain whether they did not simply miss the right spot. To improve the hit rate, the number of needle biopsies taken can be increased. Since each biopsy causes a scarf and possibly complications, this is not a preferred solution.

It is known from ultrasound imaging that a tumour gives a contrast with respect to surrounding tissue, because of its different acoustic properties, i.e. a different impedance (which depends on the velocity of sound and the specific gravity) and a different attenuation.

### SUMMARY OF THE INVENTION

Accordingly, it might be an object of the invention to provide an improved biopsy device, which device provides information about acoustic properties of a surrounding material so that the physician can be sure that the biopsy device is localized in the desired position inside the material.

These may be achieved by the subject matter according to the independent claims. Further embodiments of the present invention are described in the respective dependent claims.

Generally, a biopsy device according to the invention comprises an elongated shaft and a tip portion, and a transducer element located at the tip portion of the biopsy device, wherein the biopsy device is adapted to provide information about the acoustic properties of a material to be analysed, is proposed.

In other words, the first aspect of the present invention may be seen as based on the idea to provide a device which is adapted to take a biopsy of a material to be analysed depending on the acoustic properties (e.g. velocity) of the material whereby the information about the acoustic properties of the material is provided by the transducer element which is comprised in the biopsy device. The information about the acoustic properties may be information discriminating material, e.g. tissue of the body.

The biopsy device according to the first aspect of the invention may be adapted to take biopsies e.g. of different regions of the human body, e.g. prostate, breast/mammary gland, etc. for excluding or detecting abnormalities as e.g. cancerous lesions. Further, the biopsy device may be adapted to perform further controlling and data processing functions, e.g. analyzing functions, displaying functions, etc. The biopsy device may comprise further components, e.g. an analysing unit, controlling unit, etc.

Primarily, the biopsy device is not an imaging device, but a device for detecting various acoustic material properties, e.g. tissue properties. Anyway, a system including the biopsy device may comprise an imaging device or may be used as imaging device. Moreover, the device may not only be used to guide biopsy taking, but also to do an “acoustic biopsy”, i.e. to diagnose without removing tissue.

The biopsy device may be used in a hospital.

In the following, possible details, features and advantages of the biopsy device according to the invention will be explained on the basis of three exemplary embodiments.

The biopsy device according to the invention may be used to measure acoustic properties of the material, e.g. tissue, while inserting the tip portion of the biopsy device into the material to be analysed, which may allow for differentiating e.g. healthy tissue from cancerous tissue once the tissue has been penetrated.

For example, a cancerous tissue may have a different influence on e.g. ultrasound signals than healthy tissue. This may be seen in the detected signals.

For example, the amplitude of an acoustic signal may be a measure for absorption and scattering, whereas the delay time may be a measure for the velocity of the acoustic signal in the material, e.g. tissue. As an example, the velocity of sound in a breast tumour may be 49-90 m/s higher than in healthy tissue.

The transducer element that sends out the signal may also measure reflections of the signal in order to calculate how far the biopsy device must further be inserted to reach the tumour.

The procedure of measurement comprise signals travelling through the tissue. But also signals through the biopsy device can be analysed, that could enable to detect different tissue types close to the tip portion of the biopsy device, i.e. the biopsy device may allow measurement of the position of the tip of the biopsy device relatively to cancerous tissue.

The biopsy device may further allow measurement based on elastography, which means that the combination of images of the same anatomy in compressed and relaxed status gives a better contrast than that of the traditional ultrasound alone.

Elastography is based on a principle similar to manual palpation, in which an examiner may detect tumours because they feel harder than surrounding tissues. In elastography, a mechanical force (compression or vibration) may be applied to soft tissues, and a conventional imaging technique such as ultrasound (US) or magnetic resonance (MR) imaging may be used to create a map of soft-tissue deformation. When a discrete hard inhomogeneity, such as a tumour, is present within a region of soft tissue, a modification in the vibration amplitude will occur at its location. The cancerous tissue may behave differently than healthy tissue after being compressed and relaxed. Usually healthy tissue possesses more elasticity and relaxes faster. Since the insertion of the needle may result in local compression of the tissue, it may be useful to stop insertion procedure from time to time and let the tissue relax, and then subsequently proceed with the insertion of the needle.

Ultrasound reflection information may be taken when the needle is pushed, and when it is stopped letting the tissue to relax, then combine the measurement data and see where the boundary of e.g. a tumour is positioned with respect to the biopsy device. Finding the demarcation of different types of tissues is based on the slight acoustic impedance mismatch of the tissues (Impedance=density×acoustic velocity in the medium), causing reflection of the ultrasound. During compression both healthy and cancerous tissue are pressed, and the density of both increases. When the tissue relaxes the healthy one recovers faster due to its elasticity, therefore an increase in contrast may occur due to the temporary mismatch of the densities of the two types of tissues.

Furthermore, e.g. a tumour may also be detected without compression of tissue. By varying the angle of the needle just after insertion, the needle may be aimed at the position in which it receives a maximum in signal intensity due to reflected ultrasound.

With a biopsy device according to the first aspect of the invention, the generation of e.g. information about the acoustic properties of the material to be analyzed may be effected on the basis of e.g. high frequency data (e.g. ultrasonic data). Additionally, the generation of e.g. information about further acoustic properties and/or elastical properties of the material to be analyzed may be effected on the basis of e.g. low frequency data (e.g. low frequency ultrasound, sound, infrasound, vibration, applying pressure manually to the material to be analyzed, etc.).

The ability of providing information on different material properties can be realised by adapting the transducer elements such that they are able to detect mechanical displacements within different frequency spectra. Knowing that the response to mechanical excitation in different frequency spectra depends on physical properties of the material to be analysed, material properties correlating to elastographical properties, on the one hand, and to ultrasonic properties, on the other hand, can be derived from response signals. The mechanical excitation may be generated e.g. by the transducer element itself or manually.

Mechanical displacements may be interpreted as e.g. minimal movements or vibrations of the material, especially of cells or tissue. E.g. a displacement of cells and microscopical tissue structures may be evoked by ultrasonic pressure waves, a displacement of united macroscopical tissue structures may be caused by applying pressure to the material and slowly ranging the pressure e.g. manually or by inducing slow vibrations by the transducer elements.

In the above described first aspect of the present invention, “transducer element” may be a device, e.g. electrical, electronical or electro-mechanical, that converts one type of energy or physical attribute to another for various purposes including measurement or information transfer (e.g. pressure sensors). The transducer element of the present invention may be able to send and receive data, measure and convert different attributes and transfer and/or process information related thereto simultaneously.

A transducer element may be e.g. a small ceramic element or a single crystal. In case that the biopsy device may be disposable, low cost transducer elements may be used. This may comprise e.g. micro-machined transducer elements such as piezoelectric or capacitive micro-machined thin film transducer elements. The transducer elements may be realised in a flexible form. Further, it may be formed in various shapes, dimensions and sizes.

There may be various options to actuate the transducer element. The transducer element may send out and receive signals of various frequencies and/or amplitudes and/or time intervals.

“Material” may comprise all kind of living or dead tissue, e.g. human tissue, particularly epithelium-tissue (e.g. surface of the skin and inner lining of digestive tract), connective tissue (e.g. blood, bone tissue), muscle tissue and nervous tissue (e.g. brain, spinal cord and peripheral nervous system). “Material” may further comprise food products, biomaterials, synthetic materials, fluid or viscous substances, etc.

The distal end of the elongated shaft may be called tip. The tip may be round-shaped and/or comprise at least one edge. This edge may be formed in different shapes. The edge may be sharpened in such a way that the material, e.g. tissue, in which the biopsy device is manipulated, may be cut or easily be pierced through.

According to an aspect of the biopsy device of the present invention, the transducer element may be an ultrasound transducer element.

The transducer element may send out acoustic signals in a high frequency spectrum, which means frequencies preferably higher than 20 kHz up to 1-10 GHz.

The frequency spectrum may not be limited to a high frequency spectrum, the transducer element may further send out acoustic signals in a low frequency spectrum, which means frequencies lower than 20 kHz.

According to a further aspect of the biopsy device of the present invention, the first transducer element may be adapted to send and/or receive information.

“Sending” may signify e.g. launching any kind of signals, e.g. ultrasound signals into or on the material and/or applying mechanical pressure into or on the material.

“Receiving” may be e.g. detecting signals (e.g. reflections, resistance) of or from the material. Also the detection of higher harmonic reflected signals may be used, which may enable to improve the signal to noise ratio of a reflected signal, and in this way the detection of different material types, e.g. tissue types, may be improved.

To enable higher harmonic operation, broad bandwidth transducer elements may be used. Particularly, a thin film micro-machined transducer element with a bandwidth of >100% may be applied.

According to a first embodiment of the biopsy device, the shaft of the biopsy device may comprise a distal end which is peripherally arranged at the distal region of the shaft, wherein the shaft of the biopsy device further may comprise a planar front surface, wherein the planar front surface may be smaller than the cross section of the shaft, wherein the planar front surface may be centrally arranged relative to the shaft, wherein the transducer element may be located at the planar front surface.

Depending on the geometrical embodiment of the tip and/or the distal region of the shaft, the part of the shaft defining the planar front surface may be formed integrally with or as separate element at the shaft material, a cavity or protrusion, e.g. pin, of the shaft material or an additional object which is arranged on the shaft material.

According to an aspect of the first embodiment of the invention, the transducer element may be adapted to emit a narrow beam in the direction of the longitudinal axis of the elongated shaft of the biopsy device.

The transducer element may send out e.g. a focussed ultrasound signal in one defined direction. Accordingly, it may be possible to measure the acoustic properties only of the material which is directly located in or near the narrow beam. By this means a very high precision of the biopsy device may be reached.

A narrow beam may be technically realized with a transducer element, the length and broadness of the surface plane of which has a higher value than the wavelength of the signal sent out by the transducer element.

According to a second embodiment of the invention, the shaft of the biopsy device may comprise a distal end having an inner space and being peripherally arranged at the distal region of the shaft, wherein the transducer element may be located at a first inner sidewall of the inner space, and wherein a main signal dispersion direction of the transducer element may be orientated in the direction of a second inner sidewall opposite to the first inner sidewall of the inner space.

It is an advantage of this configuration that there may be always a reference surface at a known distance which will reflect the ultrasound, therefore the first echo recorded can give the acoustic velocity in the tissue. Moreover, it may be possible to measure continuously the acoustic properties (e.g. velocity, attenuation) of the tissue layer which is momentarily penetrated.

Such a configuration may allow to use higher ultrasound frequencies because the signal sent out by the transducer element has to penetrate just a small distance (higher frequencies lead to stronger absorption), this means that the thickness of the transducer may be decreased, facilitating the integration into the needle.

For example, for a 1 MHz piezoelectric transducer element from PVDF (polyvinylidenefluoride) the minimum thickness of the disk is around 750 micrometer without backing, which means the thickness of the complete transducer is more than 1 mm. For PZT (plumbum zirconate titanate) based compounds the compressional wave velocity is higher. In case of a PVDF transducer at 15 MHz the minimum thickness of the disc is about 50 micrometer, which enables its integration easily into the needle. The same transducer from PZT needs a 140 micron thick disc.

When ultrasound propagates through an absorbing medium with attenuation coefficient α, the initial intensity, I₀ is reduced to I_{d} at a distance d according to the expression:

I_{d}=I₀exp(−2αd)

A typical value of α for tissue may be 50 m⁻¹ at a frequency f of 5 MHz. For a biopsy device or needle with an inner diameter of 1 mm, the resulting 2-mm acoustic length would lead to a transmission of 82% according to the above-mentioned equation.

It is a further advantage of this configuration that the detected signal is relying on pulse-echo from a hard reflector and not just on random acoustical scattering in the tissue, which may it more robust.

According to an aspect of the second embodiment of the invention, the first and the second inner sidewalls define the two parallel branches of an “U”, and the transducer element is flat.

Hence, the surface of the transducer element arranged on the first inner sidewall of one branch of the “U” may be parallel to the second branch of the “U”, which may act as a hard reflector during ultrasound measurement.

The transducer element may be flat so that it may be integrated into the tip of the biopsy device so that the surface of the transducer element is parallel to the surface of the opposite metallic wall.

Preferably, the transducer may be acoustically insulated from the needle wall in order to avoid receiving signals transmitted through the needle.

According to a third embodiment of the invention, the biopsy device may comprise a plurality of transducer elements.

The biopsy device, particularly the tip portion of the shaft of the biopsy device, may comprise at least two transducer elements. Each of the transducer elements may send and/or receive signals.

There may be various options to actuate the plurality of transducer elements. One or more may send out and receive signals of various frequencies and or amplitudes and/or time intervals.

According to an aspect of the third embodiment of the invention, the plurality of transducer elements may be adapted to send out a signal, and another of the plurality of transducer elements may be adapted to detect the delay time and/or the amplitude and/or reflections of said signal.

A signal sent out by one transducer element may travel to another transducer element, where e.g. delay time and/or amplitude of said signal may be measured.

According to a further aspect of the third embodiment of the invention, the transducer elements may be orientated in different directions relative to the longitudinal axis of the elongated shaft of the biopsy device.

The transducer elements may be arranged on the shaft of the biopsy device so that the main signal dispersion direction of the transducer elements may be orientated in different directions so that it may be possible to send and detect signals to and from surrounding regions or other transducer elements of the tip portion of the biopsy device for analyzing the material, in which the tip portion of the biopsy device is inserted, as exactly as useful. Moreover, this configuration may allow to coordinate or synchronize transducer elements which are acoustically coupled with each other.

According to an aspect of any biopsy devices according to the invention, the biopsy device may be a biopsy needle or the biopsy device may comprise a hollow shaft, e.g. a canula, a trocar or a catheter, adapted to receive a needle for taking a tissue sample.

The elongated shaft of the biopsy device may comprise a bore in parallel to the longitudinal axis of the shaft. In this bore, a needle may be introduced to take a sample of the material in which the tip portion of the biopsy device has been inserted. The biopsy device may also be a canula, a trocar or a catheter.

According to a further aspect of any biopsy devices according to the invention, the biopsy device may further comprise an optical fiber, capable of emitting and receiving of light.

The biopsy device may comprise a combination of acoustic and optical sensors and actuators. The biopsy device may include at least one optical fiber, whereby the fiber may send light and receive the light after interaction with the tissue into which the tip portion of the biopsy device has been inserted.

The fiber may be connected to e.g. a console capable of probing the tissue in front of or near the biopsy device with an optical modality (e.g. reflectance spectroscopy, fluorescence spectroscopy, autofluorescence spectroscopy, differential path length spectroscopy, Raman spectroscopy, optical coherence tomography, light scattering spectroscopy, multi-photon fluorescence spectroscopy).

The optical modality may be used to e.g. to fine position the tip portion of the biopsy device in the targeted material. The optical information may be analyzed by e.g. spectral analysis. Moreover, the optical information or the analyzed optical information may be registered into an image of e.g. an additional non-invasive imaging modality.

A system of positioning a biopsy device according to the invention is proposed. Generally, the system comprises: A biopsy device as described above, and an analyzing unit, and a processing unit, and a display unit.

“Analysing” may be interpreted as exploration of the material referring to different characteristics, e.g. elastic properties, and detecting the presence and dimension of possible abnormalities compared with the physiological state or detecting pathological states as well as verifying that there are no abnormalities.

The “analysing unit” may receive analogous signals and convert them into digital signals as well as effect analysing, controlling and processing functions. The analysing unit may be separated from the biopsy device or comprised in the biopsy device. The analysing unit may further comprise e.g. a controlling unit, display unit, etc. The analysing unit may be coupled via cables, electrical conductors or wireless connection with the biopsy device.

The system of positioning a biopsy device according to the second aspect of the present invention may also comprise at least one additional imaging modality, e.g. ultrasound, magnetic resonance imaging, computed tomography, X-ray, etc.

Generally, a method for positioning a biopsy device according to the invention is proposed. The method comprises the following steps: Manipulating the biopsy device in an object of interest having tissue; transmitting an ultrasound signal by means of a transducer element; receiving a signal reflected by the tissue, by means of the transducer element; obtaining information discriminating tissue in front of or near by the tip portion of the biopsy device by means of an analyzing unit; fine positioning the biopsy device by means of the information of the analyzing unit.

The steps of the method can be partially performed in an arbitrary order or in an order as described above. The biopsy device used in the method may be the biopsy device as described above with respect to the first aspect.

The biopsy device may be applied to the surface of the object of interest. The object of interest may be any kind of material, e.g. tissue, that should be analysed. For analyzing regions that are localized inside the material it may be necessary to insert a part of the biopsy device, particularly the tip portion of the biopsy device, into the material.

The process of inserting the biopsy device may be performed by a person, e.g. a physician, or automatically by means of a technical instrument. It may be necessary to monitor the process of inserting the biopsy device into the material. This may be done e.g. by additional imaging devices, e.g. ultrasound, magnetic resonance imaging, computed tomography, X-ray, etc.

In a further step, a high frequency signal, e.g. ultrasound signal may be transmitted from at least one transducer element of the biopsy device into the material to be analysed. This signal may be reflected, scattered, attenuated, delayed or changed otherwise in the material depending from the material's specific properties, e.g. elastical properties of a tissue. The resulting signal, representing the reflected high frequency signal, may be transmitted from the material to the biopsy device and received by at least one transducer element. This resulting signal comprises the information from which the structure of the material, e.g. the elastical properties of the tissue, may be obtained in a possible subsequent analysing step.

The resulting signal may be transmitted to an analyzing unit. This analyzing unit may process the received signal. The processed signal may be visualized e.g. at a display which may e.g. be a part of the analyzing unit. The processed signal may also be presented acoustically.

The visualized and/or acoustically presented signal represents information discriminating tissue in front of or near by the tip portion of the biopsy device. By means of this information it may be possible to change the position of the biopsy device relatively to the material to be analysed so that a fine positioning of the biopsy device can be reached.

The method according to the invention may further comprise an additional step of transmitting a low frequency signal, e.g. pressure, vibration, etc. from the biopsy device into or on the material that should be analysed. This signal may be reflected in or on the material depending from the material's specific elastic properties, e.g. elastic properties of a tissue. The resulting signal, representing the reflected low frequency signal, may be transmitted from the material to the biopsy device and received by at least one transducer element. This resulting signal may comprise information from which specific elastic properties of the material, e.g. the elastic properties of a tissue, may be obtained in a possible subsequent analysing step.

The adaption of the biopsy device to the surface of the object of interest and/or the insertion of the tip portion of the biopsy device into the material, the sending and/or the receiving of the high frequency signal and a possible the low frequency signal and/or the transmission of the information to the analysing unit may take place simultaneously.

The invention relates also to a computer program for an image processing device, such that the method according to the invention might be executed on an appropriate system. The computer program is preferably loaded into a working memory of a data processor. The data processor is thus equipped to carry out the method of the invention. The computer program may be stored at a computer readable medium, such as a CD-Rom. The computer program may also be presented over a network like the worldwide web and can be downloaded into the working memory of a data processor from such a network.

It has to be noted that embodiments of the invention are described with reference to different subject matters. In particular, some embodiments are described with reference to method type claims whereas other embodiments are described with reference to apparatus type claims. However, a person skilled in the art will gather from the above and the following description that, unless other notified, in addition to any combination of features belonging to one type of subject matter also any combination between features relating to different subject matters is considered to be disclosed with this application.

The aspects defined above and further aspects, features and advantages of the present invention can also be derived from the examples of embodiments to be described hereinafter and are explained with reference to examples of embodiments. The invention will be described in more detail hereinafter with reference to examples of embodiments but to which the invention is not limited.

### BRIEF DESCRIPTION OF THE DRAWINGS

FIG. 1 shows a schematic drawing of taking a biopsy via the rectum under ultrasound guidance.

FIG. 2 a shows a schematic representation of an isometric view of the tip portion of the biopsy device according to a first embodiment of the invention.

FIG. 2 b shows a schematic representation of a side view of the tip portion of the biopsy device according to the first embodiment of the invention.

FIG. 3 a shows a schematic representation of an isometric view of the tip portion of a biopsy device according to a second embodiment of the invention.

FIG. 3 b shows a schematic representation of a longitudinal cross section of the tip portion of the biopsy device; a part of the backside of the device's tip is visible, although it is not in cross section.

FIG. 3 c shows a schematic representation of a transversal cross section of the biopsy device by plane B, seen from right to left.

FIG. 3 d shows a schematic representation of a transversal cross section of the biopsy device by plane B, seen from left to right.

FIG. 4 shows a schematic representation of the tip portion according to a third embodiment of the invention.

FIG. 5 shows a schematic representation of the use of biopsy device according to the first embodiment of the invention.

FIG. 6 shows a schematic representation of a system according to the invention.

FIG. 7 shows a schematic representation of different steps of the method according to the invention.

FIG. 8 shows a schematic representation of the signal processing steps of signals received from the material, by a biopsy device according to the invention.

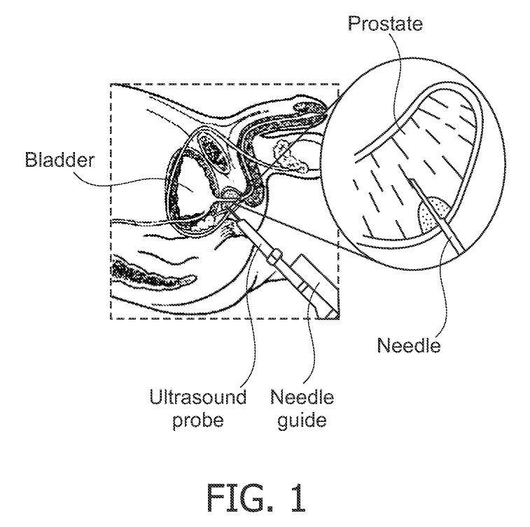

The illustration in the drawings is schematically only and not to scale. It is noted in different figures, similar elements are provided with the same reference signs.

### DETAILED DESCRIPTION OF EXEMPLARY EMBODIMENTS

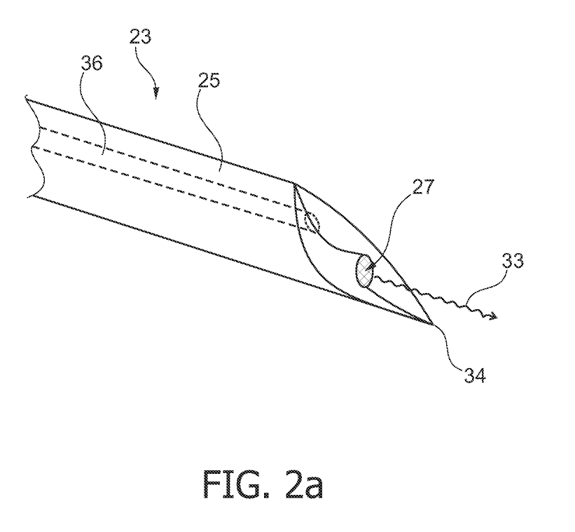

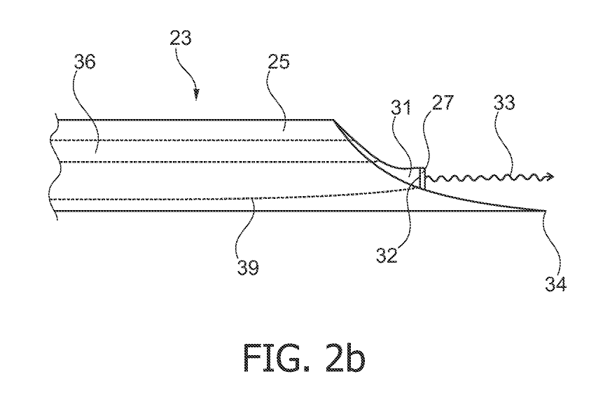

As illustrated in FIG. 2 a and FIG. 2 b, the elongated shaft 25 of the tip portion 23 of the biopsy device according to the invention comprises a bore 36 which may include e.g. a conventional needle such as a hollow metal needle for biopsy procedures, around which the material of the shaft 25 may be located.

The shaft 25 of the biopsy device comprises a distal end 34, which is peripherally arranged at the distal region of the shaft. The shaft 25 of the biopsy device further comprises a planar front surface 32 which may be centrally arranged relative to the shaft 25. The planar front surface 32 may be located on a pin 31, which is arranged on the shaft 25. The pin 31 may be a part, e.g. elevation of the shaft or a separate object, which is connected to the shaft.

A transducer element 27 for emitting and/or receiving ultrasound waves is arranged on the planar front surface 32. The wire 39 of the transducer element 27 may be embedded in the shaft 25.

The transducer element is adapted to emit a narrow beam 33 in the direction of the longitudinal axis of the elongated shaft 25 of the biopsy device.

The narrow beam may be emitted into the surrounding material.

The narrow beam may be reflected, scattered, attenuated, delayed or changed otherwise in the material depending on the material's specific properties, e.g. acoustic properties which depend on the material's elastical properties. If e.g. two neighboured portions of one material or two neighboured different materials differ in their elastic properties, the position of the tip portion of the biopsy device can be specified on the basis of the signal change received by the transducer element. In this way, with a biopsy device according to the first aspect of the invention, it may be able to establish the position of e.g. a tumour in a tissue with respect to tip of the biopsy device.

Moreover, if different characteristic values, e.g. the velocity of sound, of different materials are known in advance, the kind of material (e.g. normal tissue, cancerous tissue, etc.) may be identified depending on the signals received by the transducer element. This may allow making a diagnosis, e.g. an in vitro diagnosis, with a biopsy device according to the invention.

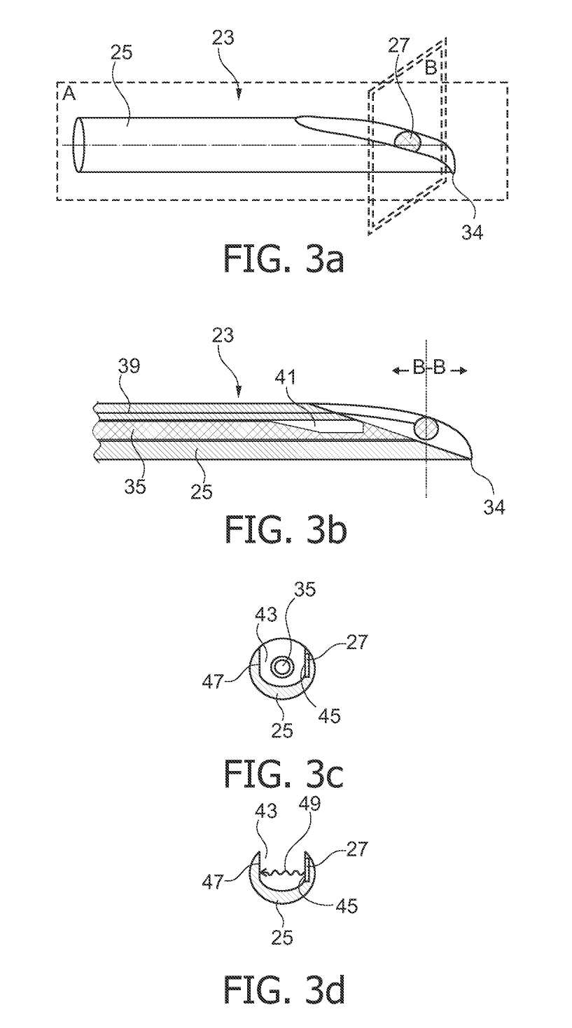

FIG. 3 a shows the tip portion 23 of a biopsy device with the elongated shaft 25 and a transducer element 27 which is arranged close to the distal end 34 of the shaft 25.

As shown in FIG. 3 b, the elongated shaft 25 of the tip portion 23 of the biopsy device according to the invention is a hollow shaft, which comprises a needle 35, such as a hollow metal needle for biopsy procedures, including a biopsy area 41.

A transducer element 27 for emitting and/or receiving ultrasound waves is arranged close to the distal end 34 of the shaft 25. The wire 39 of the transducer element 27 may be embedded in the shaft 25.

Once a suspicious spot in the tissue is detected, the needle 35 may be pushed out and withdrawn to collect a biopsy.

“B” defines an axial sectional plane.

As shown in FIG. 3 c, in the region of the distal end, the shaft 25 of the biopsy device is “U”-shaped, the two parallel branches of the “U” comprising a first inner side wall 45 and a second inner side wall 47. The shaft 25 comprises an inner space 43. The transducer element 27 is arranged in parallel to the first inner side wall 45.

FIG. 3 d shows the main signal dispersion direction 49 of the transducer element 27, which is directed from the direction of the first inner side wall 45 to the second inner side wall 47. The distance between the first inner sidewall 45 or the transducer element 27 and the second inner sidewall 47 is known. The second inner sidewall 47 may act as a hard reflector during ultrasound measurement.

The transducer element 27 must be acoustically insulated from the first inner side wall 45 and the shaft 25 in order to avoid receiving signals transmitted through the needle.

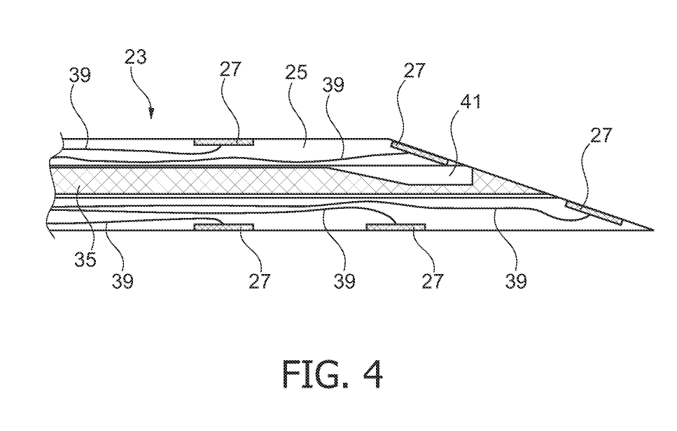

As shown in FIG. 4, the elongated shaft 25 of the tip portion 23 of the biopsy device according to the invention is a hollow shaft, which comprises a needle 35, such as a hollow metal needle for biopsy procedures, including a biopsy area 41.

Various transducer elements 27 for emitting and/or receiving ultrasound waves are arranged on the shaft of the biopsy device so that the main signal dispersion direction of the transducer elements may be orientated in different directions. The wires 39 of the transducer elements 27 may be embedded in the shaft 25.

Once a suspicious spot in the tissue is detected, the needle 35 may be pushed out and withdrawn to collect a biopsy.

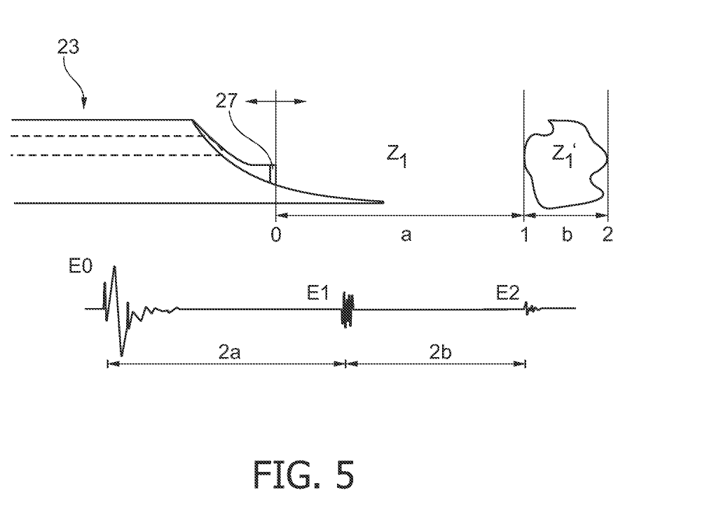

FIG. 5 shows a schematic illustration of the use of a biopsy device according to the invention to establish where the biopsy can be taken.

The distance between the transducer element 27 located at the tip portion 23 of the biopsy device and the front surface of an object of interest Z₁′ is defined as “a”, whereby “a” is variable. The distance between the front surface and the back surface of the object of interest Z₁′ is defined as “b”, whereby “b” has a fixed value. Z₁ signifies a material which is located between the transducer element 27 and the front surface of the object of interest Z₁′. The material Z₁ may be e.g. tissue, the object of interest Z₁′ may be e.g. a tumour inside the tissue.

Below the drawing of the arrangement of the biopsy device and the object of interest, an echo graph of the pulse echo awaited response is illustrated. The signals are received by the transducer element 27. “2a” signifies the double distance between the transducer element 27 and the front surface of the object of interest. “2b” signifies the double distance between the front surface and the back surface of the object of interest. “2a” is variable, “2b” has a fixed value.

E0 signifies an echo 0, E1 an echo 1 and E2 an echo 2.

The user-induced compression is due to insertion of the needle into the tissue towards the tumour. When an ultrasound image has to be taken in a relaxed position, the biopsy device is not pushed anymore into the tissue but stopped while waiting a certain interval in order to allow tissue relaxation. Since the healthy tissue relaxes faster than the tumour, the contrast in the delimitation area increases with respect to the ultrasound image taken with local compression while pushing the needle into the tissue. From the combination of the two signals (or more signals if during relaxation more measurements are performed) the distance between the needle tip and the tumour can be established as seen in the echo graph.

When echo 0 catches up with echo 1, the needle is approaching the tumor, and when subsequently echo 1 disappears, then the biopsy can be taken because the needle entered the tumor. Although it might be of a secondary importance, the posterior limit of the tumor demarcation can also be seen in echo 2, which might help in avoiding pushing the needle beyond the limits of the tumor while taking biopsy.

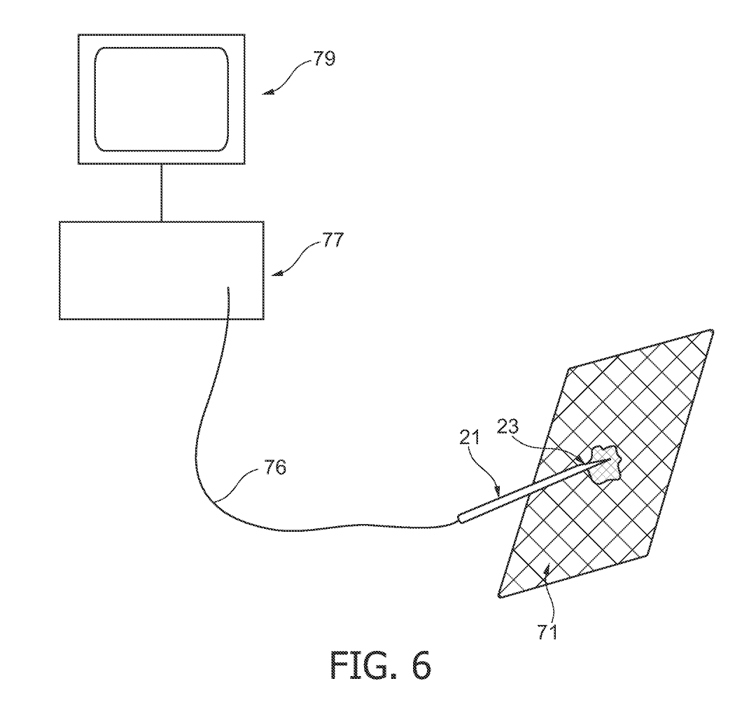

As shown in FIG. 6, the tip portion 23 of the biopsy device is located in a material to be analysed 71. The biopsy device 21 is connected to an analyzing unit 77, which is externally arranged from biopsy device 21. The analysing unit 77 is coupled via a cable 76, alternatively via electrical conductors or wireless connection, with at least one of the transducer elements 27 at the tip portion 23 of the biopsy device 21. Alternatively, the analyzing unit or a part of the analyzing unit can be comprised in the biopsy device 21 and/or in at least one of the transducer elements 27. The analysing unit 77 may be coupled with a display unit 79 via a cable, electrical conductors or wireless connection.

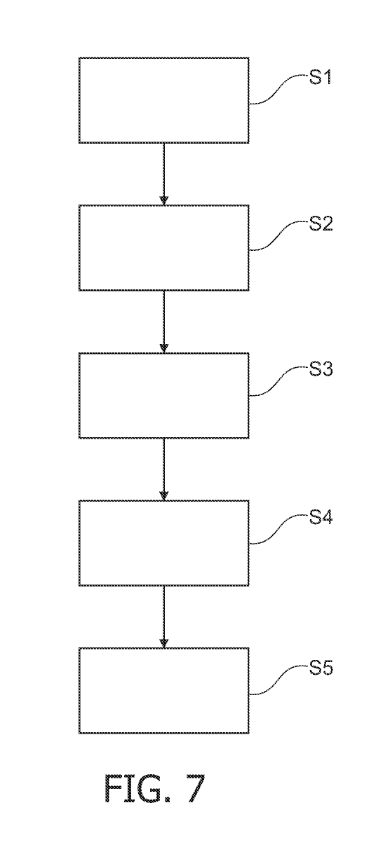

FIG. 7 shows a schematic representation of the different steps of the method according to the third aspect of the invention.

One step S1 is manipulating the biopsy device in an object of interest having tissue. This step may also include inserting a part of the biopsy device, e.g. the tip portion, into the object.

In a further step S2, an ultrasound signal is transmitted by means of at least one transducer element of the biopsy device into the object to be analysed.

A further step S3 is receiving a signal reflected by the tissue, by means of the transducer element.

Another step S4 is obtaining information discriminating tissue in front of or near by the tip portion of the biopsy device by means of an analyzing unit.

Depending on the information obtained by means of the analysing unit, there is a step of fine positioning S5 of the biopsy device.

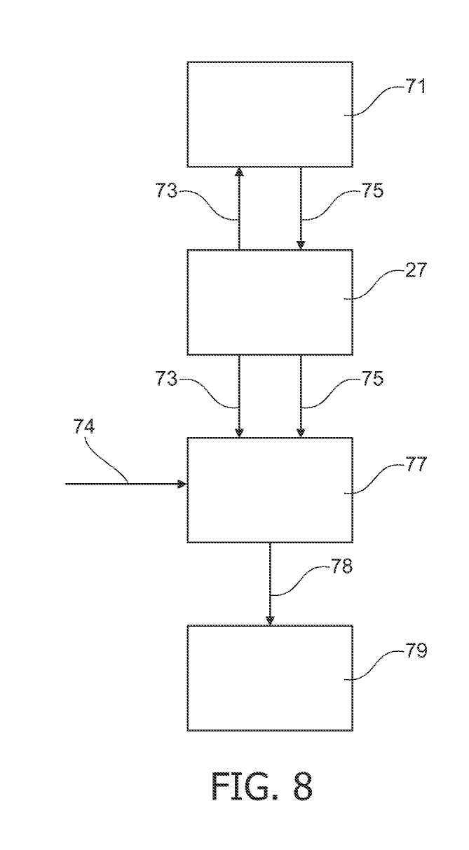

FIG. 8 shows a schematic representation of the signalling pathways and signal processing steps of the signals between a transducer element 27 and the material to be analysed 71.

An ultrasound signal 73 is transmitted from the transducer element 27 into the material to be analysed 71. This signal can be reflected at boundaries of the material depending on the material's specific structural properties. Hence, the resulting signal represents a signal reflected by the material 75, which comprises information about the architecture of the material 71. This reflected signal 75 can be transmitted from the material 71 to the transducer element 27 and can be received by the transducer element 27.

The signal reflected by the material 75 is transmitted to an analyzing unit 77 for further processing. Moreover, the ultrasound signal 73 may be also transmitted to the analyzing unit 77.

The analyzing unit is also adapted to receive further signals 74, e.g. signals from an imaging device, a controlling unit, etc. The signals received by the analyzing unit 77 can be processed and then visualised at a separate display unit 79.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

### LIST OF REFERENCE SIGNS

21 biopsy device
23 tip portion of biopsy device
25 elongated shaft
27 transducer element
31 pin
32 planar front surface
33 narrow beam
34 distal end
35 needle
36 bore
39 wire
41 biopsy area
43 inner space
45 first inner sidewall
47 second inner sidewall
49 main signal dispersion direction
71 material to be analysed
73 ultrasound signal
74 further signals
75 signal reflected by the material
76 cable
77 analyzing unit
78 information signal of analyzing unit
79 display unit
E0 echo 0
E1 echo 1
E2 echo 2
S1 manipulating biopsy device
S2 transmitting ultrasound
S3 receiving signal
S4 obtaining information
S5 fine positioning

1. A biopsy device having an elongated shaft (25) and a tip portion (23), and
a transducer element (27) located at the tip portion of the biopsy device,
wherein the biopsy device is adapted to provide information about the acoustic properties of a material to be analysed (71).

2. The device according to claim 1,
wherein the transducer element is an ultrasound transducer element.

3. The device according to claim 1,
wherein the transducer element is adapted to send and/or receive information.

4. The device according to claim 1,
wherein the shaft of the biopsy device comprises a distal end (34), which is peripherally arranged at the distal region of the shaft,
wherein the shaft of the biopsy device further comprises a planar front surface (32),
wherein the planar front surface is smaller than the cross section of the shaft,
wherein the planar front surface is centrally arranged relative to the shaft,
wherein the transducer element is located at the planar front surface.

5. The device according to claim 4,
wherein the transducer element is adapted to emit a narrow beam (33) in the direction of the longitudinal axis of the elongated shaft of the biopsy device.

6. The device according to claim 1,
wherein the shaft of the biopsy device comprises a distal end having an inner space (43) and being peripherally arranged at the distal region of the shaft,
wherein the transducer element is located at a first inner sidewall (45) of the inner space, and
wherein a main signal dispersion direction (49) of the transducer element is orientated in the direction of a second inner sidewall (47) opposite to the first inner sidewall of the inner space.

7. The device according to claim 6,
wherein the first and the second inner sidewalls define the two parallel branches of an “U”, and
wherein the transducer element is flat.

8. The device according to claim 1,
wherein the biopsy device comprises a plurality of transducer elements.

9. The device according to claim 8,
wherein one of the plurality of transducer elements is adapted to send out a signal, and another of the plurality of transducer elements is adapted to detect the delay time and/or the amplitude and/or reflections of said signal.

10. The device according to claim 8,
wherein the transducer elements are orientated in different directions relative to the longitudinal axis of the elongated shaft of the biopsy device.

11. The device according to claim 1,
wherein the biopsy device is a biopsy needle.

12. The device according to claim 1,
wherein the biopsy device comprises a hollow shaft adapted to receive a needle (35) for taking a tissue sample.

13. The device according to claim 1,
wherein the biopsy device further comprises an optical fiber, capable of emitting and receiving of light.

14. A system of positioning a biopsy device, the system comprising
a biopsy device according to claim 1, and
an analyzing unit (77), and
a processing unit, and
a display unit (79).

15. A method for positioning a biopsy device according to claim 1, the method comprising the steps of
manipulating the biopsy device in an object of interest having tissue;
transmitting an ultrasound signal by means of a transducer element;
receiving a signal reflected by the tissue, by means of the transducer element;
obtaining information discriminating tissue in front of or near by the tip portion of the biopsy device by means of an analyzing unit;
fine positioning the biopsy device by means of the information of the analyzing unit.
